(19) United States
(12) Patent Application Publication
(10) Pub. No.: US 2018/0277215 A1
(43) Pub. Date: Sep. 27, 2018

(54) STORAGE DEVICE AND ELECTRONIC APPARATUS USING THE SAME

(71) Applicant: NEW ENERGY INDUSTRY CO., LTD, Osaka (JP)

(72) Inventor: Kiyonori SHINNOU, Osaka (JP)

(21) Appl. No.: 15/466,022

(22) Filed: Mar. 22, 2017

(51) Int. Cl. G11C 16/06; A61N 5/00

(52) U.S. Cl. CPC G11C 16/06; A61N 5/00

(57) ABSTRACT

A SIM card is irradiated with quanta such as photons, electrons, quarks, or neutrinos over a predetermined cycle to be provided with quantum information, and the SIM card is inserted into a mobile information terminal having a liquid crystal screen. In response to the mobile information terminal being energized, energy of the quantum information is provided to the liquid crystal screen, causing resonance between the SIM card and the molecules (atomic nuclei) of liquid crystal, resulting in quantum energy being radiated to outside from the liquid crystal screen.

### BACKGROUND OF THE INVENTION

### 1. Field of the Invention

The present invention relates to a storage device used as a memory card or the like, and an electronic apparatus configured such that the storage device is inserted into a slot or the like of the electronic apparatus.

### 2. Description of the Related Art

Heretofore, there have been commercially available therapeutic devices for irradiating the human body with microwaves, electromagnetic waves, or the like to improve circulation of blood or to increase the effect of healing of affected areas. However, such therapeutic devices are large in scale such that, for example, a magnetron is required for microwave irradiation, and produce low effects on the inside of the human body (i.e., it is difficult for radiation to penetrate inside the human body). To address this inconvenience, the present inventor has proposed a technique disclosed in Japanese Patent No. 5684434.

In the technique disclosed in Japanese Patent No. 5684434, an alternating-current (AC) power supply is used, an external electromagnetic wave received by a mechanism similar to that of a so-called crystal radio is amplified and superimposed on an alternating current by an LC parallel resonant circuit, and only the negative component is extracted and applied to an affected area through an electrode or the like. By doing so, the technique disclosed in Japanese Patent No. 5684434 causes the outermost electron orbit of an atom of a material constituting the electrode to vary and this variation results in oscillation of protons to cause generation of quantum waves which are applied to an affected area to activate cells in the affected area.

Japanese Patent No. 5657841 discloses a sheet-type medical treatment membrane that is irradiated with the quantum waves and far infrared rays, and the sheet-type medical treatment membrane is applied to a corresponding part of the human body to achieve similar advantages. Japanese Patent No. 5727104 discloses that the quantum waves are applied to an edible oil to prevent oxidation of the edible oil.

In the techniques disclosed in Japanese Patent Nos. 5684434 and 5727104, noted above, the human body and other objects need to remain exposed to apparatuses that are smaller and easier to handle than a magnetron and the like. In the technique disclosed in Japanese Patent No. 5657841, it is only required to apply the sheet-type medical treatment membrane to the human body to achieve desired advantages. However, once the membrane is removed for, for example, bathing, a new one needs to be applied.

### SUMMARY OF THE INVENTION

It is an object of the present invention to provide a storage device with improved user convenience for a therapy using quantum energy, for example, and an electronic apparatus using the storage device.

A storage device according to an aspect of the present invention is a storage device for use in an electronic apparatus including a liquid crystal screen. The storage device stores quantum information by being irradiated with quanta over a predetermined cycle. In response to the electronic apparatus being energized, energy of the quantum information stored in the storage device resonates with molecules of liquid crystal of the liquid crystal screen to produce and externally emit quantum energy from the liquid crystal screen.

An electronic apparatus according to another aspect of the present invention comprises the storage device described above.

According to the configuration described above, the present inventor has focused on the features of a storage device in an electronic apparatus having a liquid crystal screen, the storage device cooperating with the liquid crystal screen and being provided in the form of, for example, a memory card so as to be easy to handle, and the storage device is caused to store quantum information by undergoing quantization. The storage device is preferably a flash memory.

Specifically, the quantization can be performed by application of quanta such as photons, electrons, quarks, or neutrinos over a predetermined cycle. The application of quanta can be performed by leaving the storage device under an electric potential or field that varies over the predetermined cycle for a predetermined period of time. The predetermined period of time is 2 to 3 minutes or longer, preferably, 4 to 5 minutes or longer. Under the varying electric potential or field, quarks are more likely to be produced (a larger number of quarks are produced) and neutrinos are considered to be probably produced.

The storage device that stores the quantum information in the way described above provides energy of the quantum information to the liquid crystal screen in response to the electronic apparatus being energized, causing resonance between the storage device and the molecules (in particular, atomic nuclei) of liquid crystal. Thus, quantum energy is radiated to outside from the liquid crystal screen.

When a living organism is exposed to the radiated quantum energy, tissue is activated, specifically, the electron arrangement of atoms is influenced by the quantum energy (the outermost electron orbit of a material atom is caused to vary, which results in oscillation of protons to cause generation of quantum waves). This can promote circulation of blood in, for example, an animal or can increase the effect of healing of affected areas.

In addition, the symptom of interest can be changed in accordance with the predetermined cycle. Furthermore, it is only required to subject the storage device to quantization, which is much easier than subjecting the electronic apparatus itself to quantization. Moreover, when the storage device is configured in the form of a card, the card can be inserted into a slot provided in any electronic apparatus. Hence, it is only necessary to insert the card into an electronic apparatus having a liquid crystal screen to achieve similar advantages. Accordingly, user convenience can be improved for a therapy using quantum energy, for example.

In the storage device according to the aspect of the present invention, furthermore, the predetermined cycle may be 2 to 2000 Hz, preferably, 2 to 100 Hz.

The configuration described above can achieve the effect of improving circulation of blood in a person, which may result in a stiff neck or swelling being relieved or wounds being healed quickly.

Furthermore, in the storage device according to the aspect of the present invention, irradiation of the storage device with the quanta may be performed by placing the storage device on a conductive plate and applying a negative voltage having the predetermined cycle to the conductive plate.

In the configuration described above, the storage device is subjected to quantization by placing the storage device on a conductive plate, applying a negative voltage having the predetermined cycle to the conductive plate, and applying a negative electric potential or field.

Accordingly, quanta such as photons, electrons, quarks, or neutrinos are produced on the conductive plate and act on a material atom of the storage device, enabling the storage device to store quantum information. Further, using a negative voltage as a voltage to be given can increase the amount of quantum energy.

An electronic apparatus according to still another aspect of the present invention is an information terminal into which the storage device, which is formed into a card, is inserted.

In the configuration described above, the electronic apparatus may be implemented as an information terminal such as a personal computer or a mobile phone and the storage device may be implemented as a memory card that stores a telephone number or information. Only by inserting the memory card into the electronic apparatus, it is possible to construct an electronic apparatus that can achieve the effect of promoting circulation of blood, the effect of healing of affected areas, and so on.

An electronic apparatus according to still another aspect of the present invention is portable and has a telephone function.

In the configuration described above, a user uses a mobile phone with the mobile phone kept in close contact with a portion of the user from the ear to the mouth. This allows the liquid crystal screen to come into contact with a larger portion of the human body, resulting in an increase in the effect of promoting circulation of blood or the effect of healing of affected areas.

A storage device and an electronic apparatus using the same according to aspects of the present invention are configured such that, as described above, in an electronic apparatus having a liquid crystal screen, a storage device cooperating with the liquid crystal screen is irradiated with quanta such as photons, electrons, quarks, or neutrinos over a predetermined cycle so as to be provided with quantum information. This causes energy of the quantum information to be provided to the liquid crystal screen in response to the electronic apparatus being energized, causing resonance between the storage device and the molecules of liquid crystal, resulting in quantum energy being radiated to outside from the liquid crystal screen.

Thus, when a living organism is exposed to the radiated quantum energy, tissue is activated, enabling, for example, promotion of circulation of blood in, for example, an animal or increase in the effect of healing of affected areas. Furthermore, it is only required to subject the storage device to quantization, which is much easier than subjecting the electronic apparatus itself to quantization. Moreover, when the storage device is configured in the form of a card, the card can be inserted into a slot provided in any electronic apparatus. Accordingly, user convenience can be improved for a therapy using quantum energy, for example.

### BRIEF DESCRIPTION OF THE DRAWINGS

FIG. 1 is a perspective view of an electronic apparatus according to an embodiment of the present invention;

FIGS. 2A to 2C are schematic diagrams of a method for irradiating subscriber identification module (SIM) cards with quantum information;

FIGS. 3A and 3B are waveform diagrams schematically illustrating a method for irradiating SIM cards with quantum information; and

FIG. 4 is a schematic diagram of an example of how a mobile information terminal according to an embodiment of the present invention is used.

### DESCRIPTION OF THE PREFERRED EMBODIMENTS

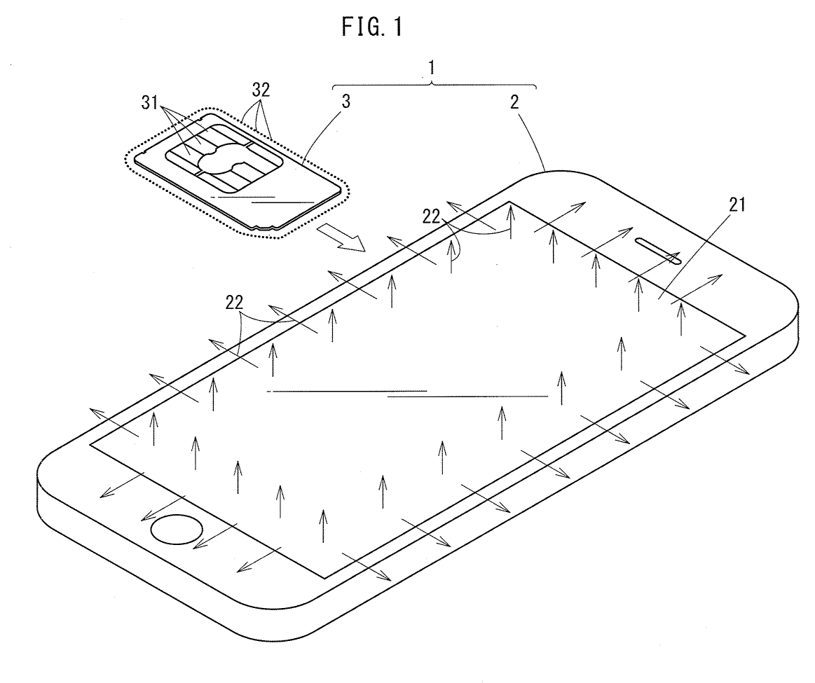

FIG. 1 is a perspective view of an electronic apparatus 1 according to an embodiment of the present invention. The electronic apparatus 1 according to this embodiment is constructed by inserting a storage device into a mobile information terminal 2. The mobile information terminal 2 is formed by a so-called smartphone. In the example in FIG. 1, the storage device is illustrated as a subscriber identification module (SIM) card 3, which is formed by a flash memory, or may be any other removable storage device such as a Secure Digital (SD) card, a Micro SD (registered trademark) card, or a Universal Serial Bus (USB) memory. The mobile information terminal 2 has on a front surface thereof a liquid crystal screen 21 having a touch panel. It may be sufficient that the electronic apparatus 1 according to the embodiment of the present invention include the liquid crystal screen 21, and the electronic apparatus 1 may be a so-called tablet terminal, a digital camera, a personal computer, or any other suitable device.

The SIM card 3 is writable, and is formed by a non-volatile electrically erasable programmable read-only memory (EEPROM) or the like that holds written information when no electric power is applied. The SIM card 3 stores the telephone number of the mobile information terminal 2, a telephone directory, contract information, and so on. The SIM card 3 is inserted into the mobile information terminal 2, thus allowing the information described above to be read to the mobile information terminal 2 via a plurality of electrodes 31 and making the mobile information terminal 2 available.

It is to be noted that, in this embodiment, the SIM card 3 for use in the mobile information terminal 2 stores quantum information 32 by being irradiated with quanta over a predetermined cycle. Accordingly, in response to the mobile information terminal 2 being energized, energy of the quantum information 32 in the SIM card 3 resonates with the molecules, mainly, atomic nuclei, of liquid crystal, enabling quantum energy 22 to be produced from the liquid crystal screen 21 and externally emitted.

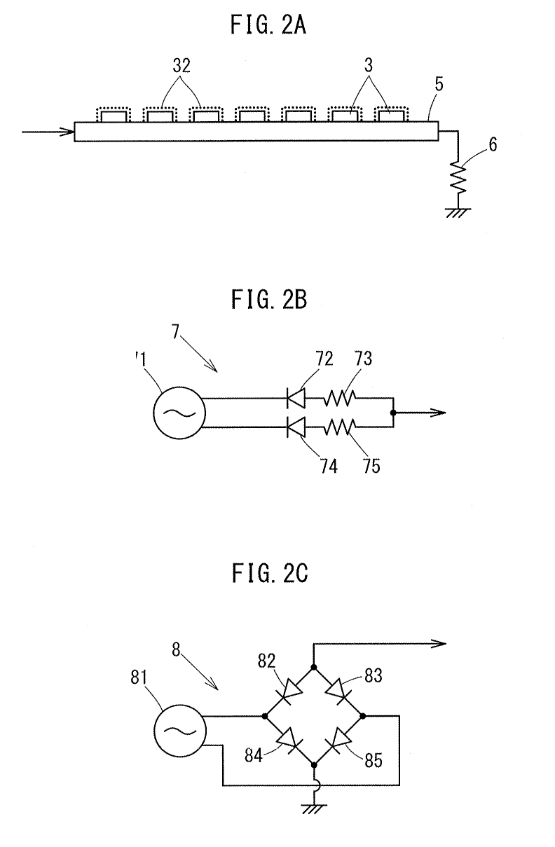

FIGS. 2A to 2C schematically illustrate a method for irradiating SIM cards 3 with quantum information 32. As illustrated in FIG. 2A, SIM cards 3 are placed on a conductive plate 5 and a negative voltage having a predetermined cycle is applied to the conductive plate 5 to apply a negative electric potential or field over a predetermined period of time, thereby allowing the SIM cards 3 to be irradiated with quantum information 32. A negative voltage is applied to the conductive plate 5 from an application circuit 7 illustrated in FIG. 2B or an application circuit 8 illustrated in FIG. 2C, thereby allowing the conductive plate 5 to provide the electric potential or field to the SIM cards 3. As illustrated in FIG. 2A, the conductive plate 5 may be grounded via a resistor 6 as high as, for example, M Ω or more.

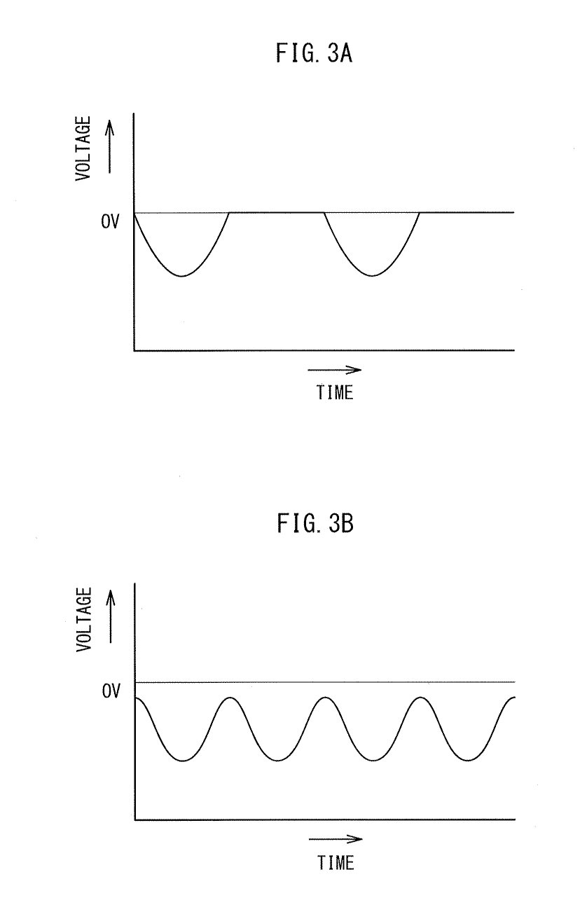

The application circuit 7 includes a series circuit of a diode 72 and a resistor 73, which is connected to one terminal of an AC power supply 71, and a series circuit of a diode 74 and a resistor 75, which is connected to another terminal of the AC power supply 71. The cathodes of the diodes 72 and 74 are connected to the AC power supply 71, and the anodes of the diodes 72 and 74 are each connected to an end of the corresponding one of the resistors 73 and 75. Accordingly, a negative half-wave voltage, as illustrated in FIG. 3A, which is extracted at the other ends of the resistors 73 and 75 is applied to the conductive plate 5. When the AC power supply 71 is a single-phase, two-wire power supply, one wire is grounded in a pole-mounted transformer or the like and the application circuit 7 ensures that a negative voltage can be extracted when it is difficult to identify which of the two wires is hot.

The application circuit 8 is formed by a diode bridge including diodes 82 to 85. The cathode of the diode 82 and the anode of the diode 84 are connected to a terminal of an AC power supply 81, and the cathode of the diode 83 and the anode of the diode 85 are connected to another terminal of the AC power supply 81. The cathodes of the diodes 84 and 85 are grounded. Accordingly, a negative full-wave voltage, as illustrated in FIG. 3B, which is extracted from the anodes of the diodes 82 and 83 is applied to the conductive plate 5.

Each of the AC power supplies 71 and 81 applies a voltage having a cycle of 2 to 2000 Hz, preferably, 2 to 100 Hz. When the AC power supplies 71 and 81 are commercial power supplies, a voltage of, for example, 100 V (effective value) is applied to the conductive plate 5 at 50 or 60 Hz. This embodiment employs the AC power supplies 71 and 81 because of their simple circuitry, and any other periodic negative voltage such as a triangular wave, rectangular wave, or pulse wave voltage may be used.

In this manner, a periodic negative voltage is applied to the conductive plate 5, thereby allowing the SIM cards 3 to be irradiated with any of 17 types of quanta, examples of which include photons, electrons, quarks, and neutrinos (above all, quarks are more likely to be produced (a larger number of quarks are produced) and neutrinos are considered to be probably produced). The SIM cards 3 are irradiated with quanta over a predetermined period of time, specifically, 2 to 3 minutes or longer, preferably, 4 to 5 minutes or longer, thereby storing the quantum information 32. A SIM card 3 that stores quantum information 32 in the way described above is inserted into a slot in the mobile information terminal 2 and then the power of the mobile information terminal 2 is turned on to energize the SIM card 3, thereby allowing energy of the quantum information 32 in the SIM card 3 to be provided to the liquid crystal screen 21. This causes the SIM card 3 and the molecules, mainly, atomic nuclei, of liquid crystal to resonate, thereby emitting the quantum energy 22 from the liquid crystal screen 21 to outside.

When a living organism is exposed to the emitted quantum energy 22, tissue is activated, specifically, the electron arrangement of atoms is affected (the outermost electron orbit of a material atom is caused to vary, which results in oscillation of protons to cause generation of quantum waves). This can promote circulation of blood in, for example, an animal or can increase the effect of healing of affected areas. In addition, the symptom of interest can be changed in accordance with the predetermined cycle.

Furthermore, it is only required to subject the SIM card 3 to quantization, which is much easier than subjecting an electronic apparatus such as the mobile information terminal 2 itself to quantization. Moreover, when a storage device is configured in the form of a card such as the SIM card 3, the card can be inserted into a slot provided in any electronic apparatus. Hence, it is only necessary to insert the card into an electronic apparatus having the liquid crystal screen 21 to achieve similar advantages. Accordingly, user convenience can be improved for a therapy using quantum energy, for example.

In addition, the predetermined cycle may be 2 to 2000 Hz, preferably, 2 to 100 Hz, thereby, according to an experiment carried out by the present inventor, achieving the effect of improving circulation of blood in a person, which may result in a stiff neck or swelling being relieved or wounds being healed quickly. According to the experiment carried out by the present inventor, in particular, in the case of a cycle of 2 to 100 Hz, the effect has been observed in the liquid crystal screen 21 in a range from a smartphone screen with a size of several inches to a television screen with a size of 30 inches. In a television set, a B-CAS (BS Conditional Access Systems Co., Ltd.) card is used in place of the SIM card 3.

The electronic apparatus 1 may be implemented as a personal computer or the like. However, the electronic apparatus 1 is implemented as the mobile information terminal 2, such as a mobile phone, and the storage device is implemented as the SIM card 3, which is configured to store telephone numbers or information. This allows realization of an electronic apparatus capable of achieving the effect of promoting circulation of blood, the effect of healing of affected areas, and so on only by inserting the SIM card 3 into the electronic apparatus.

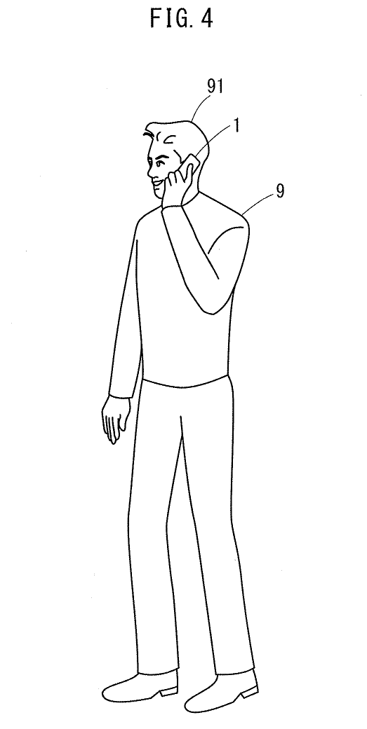

The electronic apparatus 1 may also be implemented as a portable device such as a tablet terminal with the SIM card 3 inserted or a notebook personal computer with a memory card inserted. However, the electronic apparatus 1 is preferably implemented as the mobile information terminal 2 having a telephone function because, as illustrated in FIG. 4, a user 9 uses the mobile information terminal 2 with the mobile information terminal 2 kept in close contact with a portion of the head 91 of the user 9 from the ear to the mouth, thereby allowing the liquid crystal screen 21 to come into contact with a larger portion of the human body, resulting in an increase in the effect of promoting circulation of blood or the effect of healing of affected areas.

For the irradiation of the SIM card 3 with quanta such as photons, electrons, quarks, or neutrinos, either a positive or negative voltage may be applied to the conductive plate 5. However, according to an experiment carried out by the present inventor, when a positive voltage is applied, that is, when the polarities of the diodes 72 and 74 or 82 to 85 in FIG. 2B or 2C are reversed, the quantum information 32 provided to the SIM card 3 is reduced compared to when a negative voltage is applied. Hence, it is very efficient to apply a negative voltage in the manner as illustrated in FIGS. 2A to 2C and FIGS. 3A and 3B.

1. A storage device for use in an electronic apparatus including a liquid crystal screen, wherein
the storage device accumulates quanta by being irradiated with the quanta over a predetermined cycle, and
in response to the electronic apparatus being energized, energy of the quanta accumulated in the storage device resonates with molecules of liquid crystal of the liquid crystal screen to produce and externally emit quantum energy from the liquid crystal screen.

2. The storage device according to claim 1, wherein the predetermined cycle is 2 to 2000 Hz.

3. The storage device according to claim 1, wherein the predetermined cycle is 2 to 100 Hz.

4. The storage device according to claim 1, wherein the storage device comprises a flash memory.

5. The storage device according to claim 2, wherein irradiation of the storage device with the quanta is performed by placing the storage device on a conductive plate and applying a negative voltage having the predetermined cycle to the conductive plate.

6. An electronic apparatus comprising
the storage device according to claim 1.

7. An electronic apparatus comprising
an information terminal into which the storage device according to claim 1 is inserted, the storage device being formed into a card.

8. An electronic apparatus comprising
an information terminal into which the storage device according to claim 1 is inserted, the storage device being formed into a card,
wherein the information terminal has a telephone function.

9. The storage device according to claim 1, wherein the quanta include photons, electrons, quarks, or neutrinos.

10. A method comprising:
placing a non-volatile storage device on a conductive plate;
applying, for a period of at least 2 minutes, a negative component of a voltage of substantially 100 V at a cycle of 2 to 2000 Hz to the non-volatile storage device placed on the conductive plate; and
after the applying, mounting the non-volatile storage device in an electronic apparatus including a liquid crystal screen.

11. The method according to claim 10,
wherein the non-volatile storage device accumulates quanta including photons, electrons, quarks, or neutrinos, the quanta being accumulated as a result of the applying the negative component of the voltage of substantially 100 V at the cycle of 2 to 2000 Hz to the non-volatile storage device placed on the conductive plate, and
wherein the method further comprises causing, by energizing the electronic apparatus, energy of the quanta accumulated in the non-volatile storage device to resonate with molecules of liquid crystal of the liquid crystal screen to produce and externally emit quantum energy from the liquid crystal screen.
